SEQUENCE LISTING (4 sequences)

SEQ ID NO: 1 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 2 - protein, 188 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 3 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 4 - protein, 125 aa, Homo sapiens
MARLALSPVPSHWMVALLLLLSAEPVPAARSEDRYRNPKGFSTLAQLKQRNTLKDGIIMIQTLLIILFIIVPIFLLLDKDDSKAGMEEDHTYEGLDIDQTATYEDIVTLRTGEVKWSVGEHPGQE

(19) United States
(12) Patent Application Publication
(10) Pub. No.: US 2002/0150573 A1
(43) Pub. Date: Oct. 17, 2002

(54) Anti-Igalpha-Igbeta antibody for lymphoma therapy

(75) Inventor: Michel Nussenzweig, New York, NY (US)

Correspondence Address: DARBY & DARY P.C., 805 Third Avenue, New York, NY, 10022, US

(73) Assignee: THE ROCKEFELLER UNIVERSITY

(21) Appl. No.: 10/038,107

(22) Filed: Nov. 9, 2001

Related U.S. Application Data
(60) Provisional application No. 60/247,079, filed on Nov. 10, 2000.

(51) Int. Cl. A61K 39/395; C07H 21/04; C12P 21/02; C12N 5/06; C07K 16/42

(52) U.S. Cl. 424/131.1; 536/23.53; 435/320.1; 435/326; 530/387.2

(57) ABSTRACT

The present invention provides a novel therapeutic anti-Igα-Igβ antibody that binds to an external membrane domain binding region of the Igα-Igβ heterodimer present on the cell surface of B cells. The present invention further contemplates nucleic acid sequences, host cells, and methods of producing the antibody protein. Additionally, pharmaceutical compositions comprising the anti-Igα-Igβ antibody or expression vector encoding the antibody are contemplated. Methods of inducing B cell elimination and treating a condition of inappropriate B cell activity also are contemplated.

### PRIORITY

This application claims priority under 35 U.S.C. §119 from provisional patent application Serial No. 60/247,079 filed Nov. 10, 2000; which is hereby incorporated by reference in its entirety.

### FIELD OF THE INVENTION

The present invention relates to a composition and method of inducing B cell elimination. This method can be used in the treatment of B cell lymphoma, such as chronic lymphocytic leukemia.

### BACKGROUND OF THE INVENTION

Bone marrow produces all the blood cells: red blood cells, white blood cells (also called leukocytes), and platelets. The most abundant cell type of the white blood cells are the lymphocytes and the most abundant lymphocytes are B lymphocytes (B cells) and T lymphocytes (T cells).

B cells express large quantities of antigen receptors (B cell receptor or BCR) on the cell surface after completion of light chain recombination. BCRs have two functions: (1) bind and take up antigens for presentation to T cells and (2) transmit signals that regulate B cell, development. Normal expression of the BCR requires the association of the antigen binding subunit, membrane IgM (mIgM), with the signaling component, the Igα-Igβ heterodimer. Igα-Igβ are linked by a disulfide bond. Upon binding of the BCR to an antigen, the bound antigen molecules are engulfed into the B cell by receptor-mediated endocytosis and then digested into fragments. The fragments are then expressed on the cell surface nestled inside a class II histocompatibility molecule. Helper T cells specific for this structure (i.e., with complementary TCRs) bind the B cell and secrete lymphokines that stimulate the B cell to enter the cell cycle and develop, by repeated mitosis, into a clone of cells with identical BCRs. B cells then switch from synthesizing their BCRs as integral membrane proteins to a soluble version and differentiate into plasma cells that secrete these soluble BCRs (antibodies). Expression of the Igα and Igβ is shown to be restricted to B cells and loss of BCR expression has been associated with B cell apoptosis.

B cell malignancies may arise in all lymphoid tissues where B cells are normally produced. In the case of bone marrow involvement, the transformed B cells frequently circulate through the blood and become widely disseminated throughout peripheral lymphoid tissues. However, B cell malignancies also may arise in some nonlymphoid tissues such as the thyroid, gastrointestinal tract, salivary glands and conjunctiva. Diagnosis is usually based on morphologic criteria, immunophenotyping, and the presence of monoclonal immunoglobulins in serum and/or urine and on the surface or cytoplasm of lymphocytes. Chromosome abnormalities also may be present. The transformation of B cells from small resting lymphocytes to large proliferating (transformed) lymphocytes, and the resulting displacement of normally functioning cells in the bone marrow and other lymphoid tissues relate to the clinical features of these disease states.

B cell lymphomas, such as chronic lymphocytic leukemia (CLL), express high levels of BCRs. Currently, there are no effective curative therapies for B cell lymphomas. Anti-idiotypic antibodies may be used to treat CLL. However, the antibodies must be tailored to individual patients making this approach impractical. Cross-linking the BCR with anti-Ig antibodies also can cause B cell apoptosis. However, large quantities of anti-Ig antibodies may be needed to produce an effect, due to the large quantity of circulating IgM in the serum that may interfere with the cross-linking. Additionally, there is a potential for immune complex formation and organ damage as a result of the immune complex deposition.

The present invention addresses these deficiencies by providing a strategy to develop and use a therapeutic antibody specific for Igα-Igβ, and in particular describes a method for treating B cell lymphomas with this antibody and protocol.

### SUMMARY OF THE INVENTION

The present invention provides a strategy and therapeutic antibody for eliminating B cell receptor-bearing classes of B cells from subjects in need of such treatment. In a first aspect, the invention provides an anti-Igα-Igβ antibody. This antibody binds an external membrane domain binding region of a mammalian Igα-Igβ complex. Binding of the antibody to a B cell induces B cell elimination. Preferably, the anti-Igα-Igβ antibody is human, or humanized.

Also provided is a nucleic acid encoding the anti-Igα-Igβ antibody of the invention, as well as an expression vector comprising the nucleic acid operably associated with an expression control sequence, and a host cell transfected with the expression vector.

Host cells of the invention are particularly useful for producing an anti-Igα-Igβ antibody, which comprises isolating the antibody from the host cell cultured under conditions that permit antibody expression.

Because of their ability to eliminate BCR-bearing B cells, the antibodies of the invention can be prepared in a pharmaceutical composition comprising a pharmaceutically effective amount of the antibody, wherein the pharmaceutically effective amount is sufficient to induce B cell elimination, and a pharmaceutically acceptable carrier or excipient. The pharmaceutical composition can be used in a method of eliminating a BCR-bearing B cell. This method comprises contacting the B cell with an amount of the pharmaceutical composition effective to induce elimination of B cells.

The invention further advantageously provides a method for treating a condition of inappropriate B cell activity by inducing elimination of B cells of a subject in need of such treatment. This method comprises administering to the subject an amount of the pharmaceutical composition effective to induce elimination of BCR-bearing B cells of the subject. For example, this method is effective for treating a subject suffering from a B cell lymphoma leukemia, particularly chronic lymphocytic leukemia (CLL).

Also provided is a related method of eliminating tumor cells of a B cell lymphoma. This method comprises contacting the B cell lymphoma cells with an amount of the antibody effective to eliminate the B cell lymphoma cells, particularly when the B cell lymphoma is CLL.

In addition to the antibody-containing pharmaceutical composition, the invention provides a pharmaceutical composition comprising an expression vector of the invention in an amount effective to express a therapeutically effective amount of the antibody effective to eliminate B cells in vivo. A related method for treating a condition of inappropriate B cell activity by eliminating B cells of a subject in need of such treatment comprises administering an amount of an expression vector pharmaceutical composition effective to eliminate B cells of the subject.

In a related aspect, the invention includes reagents useful for generating the antibodies. Thus, the invention provides a host cell transfected with an expression vector comprising a nucleic acid encoding Igα operably associated with an expression control sequence and transfected with an expression vector comprising a nucleic acid encoding Igβ operably associated with an expression control sequence. These host cells are useful in a method for producing a Igα-Igβ heterodimer protein. This method comprises culturing the host cell under conditions that permit expression of a Igα-Igβ heterodimer protein. Preferably, the Igα and Igβ are each expressed as a fusion protein. More preferably, the Igα and Igβ are disulfide cross-linked to each other to form a heterodimer.

Igα/Igβ products are useful in a method for producing anti-Igα-Igβ antibody. This method comprises immunizing an animal with an amount of Igα, Igβ, Igα and Igβ, or an Igα-Igβ heterodimer with an adjuvant to produce an anti-Igα-Igβ antibody, wherein the animal is a different species than the Igα-Igβ species. For example, in a preferred embodiment the animal is a mouse and the Igα-Igβ is human. In a specific embodiment, the mouse is a xenograft mouse that has a human immune system.

### DETAILED DESCRIPTION OF THE INVENTION

The present invention provides a novel therapeutic anti-Igα-Igβ antibody that binds to an external membrane domain binding region of the Igα-Igβ heterodimer present on the cell surface of B cells. Binding of the antibody to the complex induces B cell elimination. In a specific embodiment of the invention, the anti-Igα-Igβ binds from about amino acid 1 to about amino acid 112 of Igα (SEQ ID NO:1) and from about amino acid 1 to about amino acid 129 of Igβ (SEQ ID NO:3). The present invention further contemplates nucleic acid sequences, host cells, and methods of producing the antibody protein. Additionally, pharmaceutical compositions comprising the anti-Igα-Igβ antibody or expression vector encoding the antibody are contemplated. Methods of inducing B cell elimination and treating a condition of inappropriate B cell activity also are contemplated.

### Definitions

If appearing herein, the following terms shall have the definitions set out below.

The term “B cell” refers to B lymphocytes. Lymphocytes recognize and respond to foreign antigens by the production of antibodies. “Immature B cells” refers to newly produced IgM-bearing B cells that do not proliferate or differentiate in response to antigens. “Mature B cells” refers to IgM- and IgD-bearing B cells that may proliferate and/or differentiate in response to antigens.

Most mature B cells are resting when they are obtained from spleen, lymph node, or other lymphatic tissue of normal individuals. Normal individuals are individuals who do not have any B cell cancer (B cell lymphoma; myeloma or plasmacytoma; leukemia, etc.), who have normal immune function, and who are not suffering from an infection or inflammatory condition. Normal resting, mature B cells represent B cells that are not actively replicating. They can be isolated from the spleen, lymph nodes, or peripheral blood. Thus, some of them are circulating and can be readily isolated from humans. The tonsil is a lymphoid organ that contains B cells, which may be isolated from humans as well. Normal B cells are available in tonsil tissue isolated from humans who are not infected with EBV or other viruses.

The term “B cell activation” refers to the stimulation of resting B cells into the cell cycle. Stimulation generally, but not exclusively, results from binding of an antigen to membrane Ig on B cells. Antigen binding initiates proliferation, resulting in expansion of the clone, and differentiation, resulting in progeny of the B cell that actively secrete antibodies of different heavy chain isotypes or that become memory cells.

The term “inappropriate B cell activation” refers to B cell differentiation or proliferation that is increased or otherwise different from B cell differentiation or proliferation in a control sample.

The term “B cell elimination” refers to various methods that lead to B cell death or removal from the system. B cell elimination may be produced by mechanisms such as, but not limited to, apoptosis, complement mediated lysis, cell mediated cytotoxicity, and clearance of cells by phagocytosis. Additionally, B cell elimination may be induced by increasing B cell proliferation to increase susceptability to therapeutic compounds that are more efficacious in rapidly diving cells.

The term “selective” refers to an anti-Igα-Igβ antibody that recognizes and binds the external domain binding region of the Igα-Igβ complex on B cells and stimulate apoptosis.

The term “apoptosis” refers to the programmed death of a cell.

The term “phagocytosis” refers to the ingestion of particulate matter by a cell prior to degradation of the particulate.

The term “complement system” refers to a family of serum proteins that can be activated by a proteolytic cascade to generate effector molecules. The complement system mediates many of the cytolytic and inflammatory effects associated with antibody interactions. The term “complement mediated lysis” refers to the degradation of cells produced by the components of the complement system.

The term “chronic lymphocytic leukemia” and “CLL” refers to a disorder of morphologically mature but immunologically less mature lymphocytes and is manifested by progressive accumulation of these cells in the blood, bone marrow, and lymphatic tissues.

In a specific embodiment, the term “about” or “approximately” means within 20%, preferably within 10%, and more preferably within 5% of a given value or range. Alternatively, particularly in the measurement of biological processing, the term “about” or “approximately” means within an order of magnitude, preferably within a factor of 2, of a given value, e.g., a concentration of a compound that causes a half-maximal biological effect. Thus, the term “about” or “approximately” means that a value can fall within a scientifically acceptable error range for that type of value, which will depend on how quantitative a measurement can be given the available tools.

As used herein, the term “isolated” means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate unrelated materials, i.e., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

### Igα-Igβ Heterodimer

The terms “Igα” and “Igβ” refers to members of the immunoglobulin (Ig) superfamily which are associated with membrane Ig to form BCR. BCR, present on the extracellular surface of the B cell, recognizes antigens present in the system. Igα and Igβ are the signaling components of the BCR. The cytoplasmic tails comprise an immune receptor tyrosine activating motif (ITAM), which activate src and sky family tyrosine kinases (Reth, Curr. Opin. Immunol., 1994, 12:196-201; Pleiman et al. Immunol. Today, 1994, 15:393-398).

Two variants of both the Igα and Igβ human proteins have been isolated and sequenced. The major form of both proteins include additional amino acids that are not present in the minor variant. The major form of Igα is 194 amino acids in length (SEQ ID NO:1; Genbank accession number NM_{—}001783) whereas the minor variant is 156 amino acids in length (SEQ ID NO:2; Gebank accession number NM_{—}021601). The major form of Igα contains a 114 base pair in-frame insertion in the coding sequence, which results in an additional 38 amino acids in the extracellular domain of the protein. Additionally, one amino acid position is changed from a glycine to a glutamic acid. As a result of the deletion, 2 out of 3 cysteine residues and 3 out of 6 potential N-glycosylation sites are deleted. In one embodiment, the Igα of the present invention is depicted in SEQ ID NO:1.

The major form of Igβ is 201 amino acids in length (SEQ ID NO:3; Genbank accession number NM_{—}000626) whereas the minor variant is 97 amino acids in length (SEQ ID NO:4; Gebank accession number NM_{—}021602). The major form of Igβ contains a 312 base pair in-frame insertion in the coding sequence, which results in an additional 104 amino acids in the extracellular domain of the protein. As a result of the deletion, all 5 cysteine residues and all 3 potential N-glycosylation sites are deleted. In one embodiment, the Igβ of the present invention is depicted in SEQ ID NO:3.

The Igα and Igβ are associated by an extracellular disulfide bond to produce a heterodimer. The “extracellular domain” refers to regions of the chains located on the outside of the B cell. The amino acids that comprise this domain are from about amino acid 1 to about amino acid 112 of Igα (SEQ ID NO:1) and from about amino acid 1 to about amino acid 129 of Igβ (SEQ ID NO:3). Igα and Igβ may be derived from any mammalian source, preferably human but also including chimpanzee, ape, monkey, canine, feline, murine, racine, ovine, caprine, bovine, equine, avian, etc. Thus the invention advantageously provides a method for enhancing B cell elimination elicited by this therapeutic antibody in vivo in a human or other animal, e.g., of species set forth above.

The therapeutic anti-Igα-Igβ antibody can act by disrupting activation of BCR receptors present in B cells; inducing B cell apoptosis, by eliciting endogenous immune mechanisms (opsonization, antibody-mediated cellular activation); complement activation; and direct delivery of an active agent such as a radionuclide or a toxin.

Antibodies to Igα or Igβ have been difficult to produce due to the use of crude preparations of peptide fragments. Additionally, the individual components are difficult to express in a stable fashion. The present invention contemplates novel methods of producing a Igα-Igβ heterodimer. This heterodimer may be used to immunize animals for the production of anti-Igα-Igβ antibodies. Unexpectedly, antibodies elicited against the recombinant heterodimer are therapeutically effective.

The present invention discloses the production of a heterodimer of Igα-Igβ, including the disulfide link, particularly using recombinant technology. Any method of producing this heterodimer is contemplated by the present invention. In a preferred embodiment, a host cell is transfected (i) with an expression vector comprising a nucleic acid encoding Igα (which includes full length, the extracellular domain, or a fusion construct comprising the extracellular domain) operably associated with an expression control sequence and (ii) with an expression vector comprising a nucleic acid encoding Igβ (which includes full length, the extracellular domain, on a fusion construct comprising the extracellular domain) operably associated with an expression control sequence. The nucleic acids encoding Igα and Igβ can be on separate vectors or the same vector. If on the same vector they can each be operatively associated with the same or different (preferably the same) promoters, or alternatives arranged in a bicistronic construct with one promoter at the 5′ end and an internal ribosome entry site (IRES) between the coding sequences. The cells are then cultured under conditions that permit expression of both proteins. The proteins are then post-translationally modified to produce the Igα-Igβ heterodimer. The expression vectors may encode a soluble or membrane form of Igα and/or Igβ by modification of the transmembrane and/or cytoplasmic domains of the protein sequence.

In a preferred embodiment, Igα or Igβ, or both may be expressed as a fusion protein, where the protein is fused to a fusion partner, such as but not limited to, an immunoglobulin constant region domain (Ig), a FLAG tag, a HIS tag, a myc tag, a heterologous signal peptide, a yeast peptide, and the like. Fusion polypeptides that comprise a signal sequence domain can be used to target the fusion polypeptide for secretion by a host cell into the culture medium for extraction and purification. Fusion polypeptides comprising a transmembrane domain can be used to target fusion polypeptides for expression on the cell surface.

The tags may be used for isolation, detection, or purification of the proteins, using methods that are well known in the art. Such methods include, but are not limited to, affinity chromatography, ELISA, Western blot, FACS, and immunohistochemistry. Tags may be (but need not be) removed by enzymatic digestion prior to immunization of an animal for antibody production.

In a specific embodiment, Igα and Igβ are each expressed as a fusion construct in which the extracellular domain, as set forth above, is fused with an Ig domain. Such a construct yields a soluble protein, including after crosslinking. A soluble Igα/Ig-Ig/Igβ heterodimer has enhanced immunogenicity, and elicits a stronger specific immune response against BCR-bearing B cells.

Heterodomain formation and disulfide crosslinking can be achieved by contacting approximately equimolar concentrations of Igα and Igβ under oxidizing conditions. Preferably, the reactive cysteines are asymmetrically activated so that Igα preferentially or exclusively disulfide cross-links with Igβ rather than itself, and Igβ preferably or exclusively disulfide cross-links with Igα rather than itself.

### Antibodies

The term “antibody” is used in the broadest sense and specifically covers monoclonal antibodies (including full length monoclonal antibodies), polyclonal antibodies, multispecific antibodies (e.g., bispecific antibodies), and antibody fragments, so long as they exhibit the desired biological activity. In a specific embodiment, the anti-Igα-Igβ antibody recognizes the external membrane binding domains of the Igα and Igβ proteins present on the cell. In a further embodiment, the antibody recognizes from about amino acid 1 to about amino acid 112 of Igα or from about amino acid 1 to about amino acid 129 of Igβ or an epitope generated from both chains. Preferably, Igα and Igβ have the sequences as depicted in SEQ ID NOs:1 and 3, respectively. Antibodies of the invention are characterized by the ability to eliminate BCR-bearing B cells, which is the “desired biological activity”.

“Antibody fragments”, as defined for the purpose of the present invention, comprise a portion of an intact antibody, generally including the antigen binding or variable region of the intact antibody or the Fc region of an antibody which retains FcR binding capability. Examples of antibody fragments include linear antibodies; single-chain antibody molecules; and multispecific antibodies formed from antibody fragments. The antibody fragments preferably retain at least part of the hinge and optionally the CH1 region of an IgG heavy chain. More preferably, the antibody fragments retain the entire constant region of an IgG heavy chain, and include an IgG light chain.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 1984, 81:6851-6855; Neuberger et al., Nature, 1984, 312:604-608; Takeda et al., Nature, 1985, 314:452-454; International Patent Application No. PCT/GB85/00392).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature, 31986, 21:522-525; Riechmann et al, Nature, 1988, 332:323-329; Presta, Curr. Op. Struct. Biol., 1992, 2:593-596; U.S. Pat. No. 5,225,539.

According to the invention, Igα, Igβ, or Igα-Igβ heterodimer polypeptide produced recombinantly or by chemical synthesis, and fragments or other derivatives or analogs thereof, including fusion proteins, may be used as an immunogen to generate antibodies that recognize the Igα, Igβ, or Igα-Igβ heterodimer polypeptide. As noted above, such antibodies include but are not limited to polyclonal, monoclonal, chimeric, single chain, Fab fragments, and an Fab expression library. Preferably the antibodies are generated against a soluble Igα, Igβ, or Igα-Igβ heterodimer construct, e.g., made by fusing the extracellular domains (as defined above) to another protein, such as an immunoglobulin Fc domain.

Various procedures known in the art may be used for the production of polyclonal or monoclonal antibodies to Igα, Igβ, or Igα-Igβ heterodimer polypeptide or fragment, analog, or derivative thereof. For the production of antibody, various host animals can be immunized by injection with the Igα, Igβ, or Igα-Igβ heterodimer polypeptide, or a derivative (e.g., fragment or fusion protein) thereof, including but not limited to rabbits, mice, rats, sheep, goats, etc. Preferably the animal is a chimeric non-human mammal, such as a mouse, comprising a human immune system graft, so that the antibodies are human antibodies generated in the non-human (e.g., murine) genetic background (see below). In one embodiment, the Igα, Igβ, or Igα-Igβ heterodimer polypeptide or fragment thereof (particularly a fragment from the extracellular domain) can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

Any technique that provides for the production of antibody molecules by continuous cell lines in culture may also be used. These include but are not limited to the hybridoma technique originally developed by Kohler and Milstein (Nature 256:495-497, 1975), as well as the trioma technique, the human B cell hybridoma technique (Kozbor et al., Immunology Today 1983, 4:72; Cote et al., Proc. Natl. Acad. Sci. U.S.A. 1983, 80:2026-2030), and the EBV-hybridoma technique to produce human monoclonal antibodies (Cole et al., in Monoclonal Antibodies and Cancer Therapy, Alan R. Liss, Inc., pp. 77-96, 1985). In an additional embodiment of the invention, monoclonal antibodies can be produced in germ-free animals (International Publication No. WO 89/12690). In fact, according to the invention, techniques developed for the production of “chimeric antibodies” or “humanized antibodies” (U.S. Pat. No. 4,816,567; International Application No. PCT/GB85/00392; Morrison et al., J. Bacteriol. 1984, 159:870; Neuberger et al., Nature 1984, 312:604-608; Takeda et al., Nature 1985, 314:452-454) by splicing or grafting sequences from a mouse antibody molecule specific for an Igα, Igβ, or Igα-Igβ heterodimer polypeptide, particular CDR sequences, together with genes from a human antibody molecule of appropriate biological activity can be used; such antibodies are within the scope of this invention. Such human or humanized chimeric antibodies are preferred for use in therapy of human diseases or disorders (described infra).

One embodiment of the chimeric non-human mammal discussed above is a Xeno Mouse™ (Abgenix; Fremont, Calif.). In these animals, immunization produces antibodies having primate, particularly human, constant and/or variable regions. These animals are characterized by (1) being incapable of producing exdogenous immunoglobulin and (2) an exogenous immunoglobulin locus comprising (i) at least one immunoglobulin constant region, (ii) immunoglobulin sequences for the components of the variable region, and (iii) at least one intron. Methods for producing these animals are disclosed in U.S. Pat. Nos. 5,939,598 and 6,075,181 and PCT Publication WO 98/24893. These animals maybe immunized with the proposed antigen, as described in U.S. Pat. No. 6,114,598, to produce antibodies of the present invention.

According to the invention, techniques described for the production of single chain antibodies (U.S. Pat. Nos. 5,476,786 and 5,132,405 to Huston; U.S. Pat. No. 4,946,778) can be adapted to produce Igα, Igβ, or Igα-Igβ heterodimer polypeptide-specific single chain antibodies. Indeed, these genes can be delivered for expression in vivo. An additional embodiment of the invention utilizes the techniques described for the construction of Fab expression libraries (Huse et al., Science 246:1275-1281, 1989) to allow rapid and easy identification of monoclonal Fab fragments with the desired specificity for an Igα, Igβ, or Igα-Igβ heterodimer polypeptide, or its derivatives, or analogs.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Antibodies may be screened for specificity by staining human cell lines including, but not limited to, fibroblasts, epithelial cells, red blood cells, platelets, macrophages, mast cells, polymorphonuclear leukocytes, and neuronal cell lines. Many means are known in the art for detecting binding in an immunoassay and are within the scope of the present invention. For example, to select antibodies which recognize a specific epitope of an Igα, Igβ, or Igα-Igβ heterodimer polypeptide, one may assay generated hybridomas for a product which binds to an Igα, Igβ, or Igα-Igβ heterodimer polypeptide fragment containing such epitope. For selection of an antibody specific to an Igα, Igβ, or Igα-Igβ heterodimer polypeptide from a particular species of animal, one can select on the basis of positive binding with Igα, Igβ, or Igα-Igβ heterodimer polypeptide expressed by or isolated from cells of that species of animal.

In accordance with the invention, antibodies that agonize or antagonize the activity of the Igα-Igβ component of the B cell receptor can be generated. Such antibodies can be tested for their ability to eliminate B cells using the in vitro and in vivo assays described infra.

### Recombinant Technology

In accordance with the present invention there may be employed conventional molecular biology, microbiology, and recombinant DNA techniques within the skill of the art to express the Igα and Igβ constructs and heterodimers, and to express recombinant therapeutic antibodies as set forth above. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

A “vector” is a recombinant nucleic acid construct, such as plasmid, phage genome, virus genome, cosmid, or artificial chromosome, to which another DNA segment may be attached. In a specific embodiment, the vector may bring about the replication of the attached segment, e.g., in the case of a cloning vector. A “replicon” is any genetic element (e.g., plasmid, chromosome, virus) that functions as an autonomous unit of DNA replication in vivo, i.e., it is capable of replication under its own control.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell and the cell expresses a nononcogenic protein. A cell has been “transformed” by exogenous or heterologous DNA when the transfected DNA is expressed and effects a function or phenotype on the cell in which it is expressed, i.e. cell shows oncogenic properties.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is a such an element operatively associated with a different gene than the one it is operatively associated with in nature. In the context of the present invention, the vectors for expression of the estrogen receptor, transcription factor, and reporter gene operatively associated with the E-selectin promoter is heterologous to a host cell in which it is expressed, e.g., an endothelial cell.

A “gene” is used herein to refer to a portion of a DNA molecule that includes a polypeptide coding sequence operatively associated with expression control sequences. In one embodiment, a gene can be a genomic or partial genomic sequence, in that it contains one or more introns. In another embodiment, the term gene refers to a cDNA molecule (i.e., the coding sequence lacking introns). Generally, as used herein, the term “gene” refers to a coding sequence operatively associated with an expression control sequence (e.g., promoter and termination signal), which may be heterologous or homologous to be coding sequence.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

“Expression control sequences”, e.g., transcriptional and translational control sequences, are regulatory sequences that flank a coding sequence, such as promoters, enhancers, suppressors, terminators, and the like, and that provide for the expression of a coding sequence in a host cell. In eukaryotic cells, polyadenylation signals are control sequences. On mRNA, a ribosome binding site is an expression control sequence.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “operatively associated with” or “under the control” of transcriptional and translational control sequences in a cell when RNA polymerase transcribes the coding sequence into mRNA, which is then trans-RNA spliced (if it contains introns) and translated into the protein encoded by the coding sequence.

A “signal sequence” is included at the beginning of the coding sequence of a protein to be expressed on the surface or in the membrane of a cell, or that is to be secreted from the cell. This sequence encodes a signal peptide, N-terminal to the mature polypeptide, that directs the host cell to translocate the polypeptide.

Recombinant vectors can be introduced into host cells via calcium phosphate precipitation, infection/viral transformation, electroporation, lipofection, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2μ plasmid.

As used herein the term “transformed cell” refers to a modified host cell that expresses an anti-Igα-Igβ antibody expressed from a vector encoding the anti-Igα-Igβ antibody. Any cell can be used, preferably a mammalian cell.

### Expression Vectors

The nucleotide sequence coding for Igα and/or Igβ proteins or the anti-Igα-Igβ antibody can be inserted into an appropriate expression vector, i.e., a vector which contains the necessary elements for the transcription and translation of the inserted protein-coding sequence. Thus, the nucleic acid encoding the Igα and/or Igβ proteins or the anti-Igα-Igβ antibody is operatively associated with a promoter in an expression vector of the invention. Both cDNA and genomic sequences can be cloned and expressed under control of such regulatory sequences. An expression vector also preferably includes a replication origin. The expression vector may also include a sequence for a fusion tag such as, but not limited to a FLAG or HIS tag.

A wide variety of host/expression vector combinations (i.e., expression systems) may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., 1988; Gene, 67:31-40, 1988), pMB9 and their derivatives, plasmids such as RP4; phage DNAS, e.g., the numerous derivatives of phage 1, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. In addition, various tumor cells lines can be used in expression systems of the invention.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme. Host cells are used for screening or other assays, as described infra. A preferred expression host is a eukaryotic cell (e.g., yeast, insect, or mammalian cell). More preferred is a mammalian cell, e.g., human, rat, monkey, dog, or hamster cell.

A recombinant Igα, Igβ, or anti-Igα-Igβ antibody protein may be expressed chromosomally, after integration of the coding sequence by recombination. In this regard, any of a number of amplification systems may be used to achieve high levels of stable gene expression (See Sambrook et al., 1989, supra).

Yeast expression systems can also be used according to the invention to express any protein of interest. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

Expression of the protein or polypeptide may be controlled by any promoter/enhancer element known in the art, but these regulatory elements must be functional in the host selected for expression. Promoters which may be used to control gene expression include, but are not limited to, cytomegalovirus (CMV) promoter (U.S. Pat. No. 5,385,839 and No. 5,168,062), the SV40 early promoter region (Benoist and Chambon, 1981; Nature, 290:304-310), the promoter contained in the 3′ long terminal repeat of Rous sarcoma virus (Yamamoto, et al., 1980; Cell, 22:787-797), the herpes thymidine kinase promoter (Wagner et al., 1981; Proc. Natl. Acad. Sci. U.S.A., 78:1441-1445), the regulatory sequences of the metallothionein gene (Brinster et al., 1982; Nature, 296:39-42); prokaryotic expression vectors such as the β-lactamase promoter (Villa-Komaroff, et al, 1978; Proc. Natl. Acad. Sci. U.S.A., 75:3727-3731), or the tac promoter (DeBoer, et al., 1983; Proc. Natl. Acad. Sci. U.S.A., 80:21-25); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., 1985; Nature, 315:338-340; Kollias et al., 1986; Cell, 46:89-94), hematopoietic stem cell differentiation factor promoters, erythropoietin receptor promoter (Maouche et al., 1991; Blood, 15:2557), etc.

Preferred vectors, particularly for cellular assays in vitro, are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional protein can be introduced in vitro using a viral vector or through direct introduction of DNA.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., 1991; Molec. Cell. Neurosci., 2:320-330), defective herpes virus vector lacking a glyco-protein L gene (Patent Publication RD 371005 A), or other defective herpes virus vectors (PCT WO 94/21807, published Sep. 29, 1994; PCT WO 92/05263, published Apr. 2, 1994); an attenuated adenovirus vector, such as the vector described by Stratford-Perricaudet et al. (J. Clin. Invest., 90:626-630, 1992; see also La Salle et al., 1993; Science, 259:988-990); and a defective adeno-associated virus vector (Samulski et al., 1989; J. Virol., 61:3096-3101, 1987; Samulski et al., 1987; J. Virol., 63:3822-3828, 1989; Lebkowski et al., Mol. Cell. Biol., 8:3988-3996).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Adenovirus Vectors.

Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5) or adenoviruses of animal origin (see WO 94/26914). Those adenoviruses of animal origin which can be used within the scope of the present invention include adenoviruses of canine, bovine, murine (example: Mav1, Beard et al, 1980; Virology, 75:81), ovine, porcine, avian, and simian (example: SAV) origin. Various replication defective adenovirus and minimum adenovirus vectors have been described (PCT Publication Nos. WO 94/26914, WO 95/02697, WO 94/28938, WO 94/28152, WO 94/12649, WO 95/02697 WO 96/22378).

Adeno-Associated Viruses.

The adeno-associated viruses (AAV) are DNA viruses of relatively small size which can integrate, in a stable and site-specific manner, into the genome of the cells which they infect. The use of vectors derived from the AAVs for transferring genes in vitro and in vivo has been described (see PCT Publication Nos. WO 91/18088; WO 93/09239; U.S. Pat. Nos. 4,797,368; 5,139,941, EP 488 528).

Retrovirus Vectors.

In another embodiment the gene can be introduced in a retroviral vector, e.g., as described in Anderson et al., U.S. Pat. No. 5,399,346; Mann et al., 1983; Cell, 33:153; Temin et al., U.S. Pat. Nos. 4,650,764 and 4,980,289; Markowitz et al., 1988; J. Virol., 62:1120; Temin et al., U.S. Pat. No. 5,124,263; EP 453242, EP178220; Bernstein et al. 1985; Genet. Eng., 7:235; McCormick, 1985; BioTechnology, 3: 689; PCT Publication No. WO 95/07358; and Kuo et al, 1993; Blood, 82:845. The retroviruses are integrating viruses which infect dividing cells. These vectors can be constructed from different types of retrovirus, such as, HIV, MoMuLV (“murine Moloney leukaemia virus”) MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PA317 (U.S. Pat. No. 4,861,719); the PsiCRIP cell line (WO 90/02806) and the GP+envAm-12 cell line (WO 89/07150).

Lentivirus Vectors.

In another embodiment, lentiviral vectors can be used as agents for the direct delivery and sustained expression of a transgene (for a review, see, Naldini, 1988; Curr. Opin. Biotechnol., 9:457-63, see also Zufferey et al., 1998; J. Virol., 72:9873-80). Lentiviral packaging cell lines are available and known generally in the art (see Kim et al., 1998; J. Virology 72:811-816). High-titer lentivirus vectors have been found to be excellent transfection agents for protein function assays in tissue cultured cells. An example is a tetracycline-inducible VSV-G pseudotyped lentivirus packaging cell line which can generate virus particles at titers greater than 10⁶ IU/ml for at least 3 to 4 days (Kafri, et al., 1999; J. Virol., 73: 576-584). The vector produced by the inducible cell line can be concentrated as needed for efficiently transducing nondividing cells in vitro.

Non-Viral Vectors.

In another embodiment, the vector can be introduced by lipofection, as naked DNA, or with other transfection facilitating agents (peptides, polymers, etc.). Synthetic cationic lipids can be used to prepare liposomes for transfection of a gene (Felgner, et. al., 1987; Proc. Natl. Acad. Sci. U.S.A., 84:7413-7417; Felgner and Ringold, 1989; Science, 337:387-388; see Mackey, et al., 1988; Proc. Natl. Acad. Sci. U.S.A., 85:8027-8031, 1988; Ulmer et al., 1993; Science, 259:1745-1748, 1993). Useful lipid compounds and compositions for transfer of nucleic acids are described in PCT Publication Nos. WO 95/18863 and WO 96/17823, and in U.S. Pat. No. 5,459,127. Lipids may be chemically coupled to other molecules for the purpose of targeting (see Mackey, et. al., supra).

Other molecules are also useful for facilitating transfection of a nucleic acid, such as a cationic oligopeptide (e.g., PCT Publication WO 95/21931), peptides derived from DNA binding proteins (e.g., PCT Publication WO 96/25508), or a cationic polymer (e.g., PCT Publication WO 95/21931).

It is also possible to introduce the vector as a naked DNA plasmid. Naked DNA vectors can be introduced into the desired host cells by methods known in the art, e.g., electroporation, electrotransfer (PCT Publications WO 99/01157, WO 99/01158, and WO 99/01175), microinjection into cells, direct injection into tissues (U.S. Pat. Nos. 5,580,859 and 5,589,466), cell fusion, DEAE dextran, calcium phosphate precipitation, use of a gene gun, or use of a DNA vector transporter (see, e.g., Wu et al., 1992; J. Biol. Chem., 267:963-967; Wu and Wu, 1988; J. Biol. Chem., 263:14621-14624; Hartmut et al., Canadian Patent Application No. 2,012,311, filed Mar. 15, 1990; Williams et al, 1991; Proc. Natl. Acad. Sci. USA, 88:2726-2730). Receptor-mediated DNA delivery approaches can also be used (Curiel et al., 1992; Hum. Gene Ther., 3:147-154; Wu and Wu, 1987; J. Biol. Chem., 262:4429-4432).

### B Cell Elimination Assays

### In Vitro Testing

Any cell assay system that allows for assessment of B cell elimination is contemplated by the present invention. The assays can be used to confirm antibodies of the invention that specifically regulate B cell elimination. B cell elimination can occur by any of the mechanisms set forth above. For example, a non-limiting list of studies that may be used to detect B cell apoptosis includes DNA staining by propidium iodide, TUNEL labeling, in situ end labeling, Annexin V binding, caspase activity, and flow and laser cytometry. A non-limiting list of studies that may be used to detect complement mediated lysis includes CH50 and AH50 assays, measurement of complement component levels by radial immunodiffusion, and Factor B or Factor D hemolytic assay. Protein function assays, such as ³H thymidine incorporation or MZT metabolism assays reveal stimulation of the target B cells by the anti-Igα-Igβ antibody. Such techniques, as well as other techniques that may be used, are explained fully in the literature (see, e.g., Coligan, Kruisbeek, Margulies, Shevach, and Strober, Current Protocols in Immunology, (1991) John Wiley & Sons, Inc, New York). Studies may be performed on CLL cell lines and on primary CLL tumor cells from patients.

Any convenient method that permits detection of expression of an apoptosis or other indicator product is contemplated. For measurement of mRNA expression, for example, the invention provides Northern blot analysis for detecting mRNA product. The methods comprise, for example, the steps of fractionating total cellular RNA on an agarose gel, transferring RNA to a solid support membrane, and detecting a DNA-RNA complex with a labeled DNA probe, wherein the DNA probe is specific for a particular nucleic acid sequence of the product mRNA under conditions in which a stable complex can form between the DNA probe and RNA components in the sample. Such complexes may be detected by using any suitable means known in the art, wherein the detection of a complex indicates the presence of product in the sample. Comparatively, isolated RNA may be subjected to coupled reverse transcription and amplification by polymerase chain reaction (RT-PCR), using specific oligonucleotide primers that are specific for a selected sequence. Conditions for primer annealing are chosen to ensure specific reverse transcription and amplification. RT-PCR products than may be fractionated on an agarose gel, identified by base pair size, and quantified.

For measurement of protein expression, the invention provides, for example, antibody-based methods for detecting protein expression. The methods comprise the steps of detecting an antigen-antibody complex formed by contacting a sample with one or more antibody preparations, wherein each of the antibody preparations is specific for a particular polynucleotide sequence of the protein under conditions in which a stable antigen-antibody complex can form between the antibody and antigenic components in the sample. Such complexes mat be detected by using any suitable means known in the art, wherein the detection of a complex indicates the presence of the protein in the sample.

Various in vitro antibody dependent cell-mediated cytotoxicity and phagocytosis assays are available to detect antibody-mediated cell clearance (reviewed in Daeron, Ann. Rev. Immunol. 1997, 15:203-204; Ward and Ghetie, Therapeutic Immunol. 1995, 2:77-94; Ravetch and Kinet, Annu. Rev. Immunol. 1991, 9:457-492).

Typically, immunoassays use either a labeled antibody or a labeled antigenic component (e.g., that competes with the antigen in the sample for binding to the antibody). Suitable labels include without limitation enzyme-based, fluorescent, chemiluminescent, radioactive, or dye molecules. Assays that amplify the signals from the probe are also known, such as, for example, those that utilize biotin and avidin, and enzyme-labeled immunoassays, such as ELISA assays.

### In Vivo Testing Using Engrafted Animals

The present invention permits the ability of a candidate antibody to eliminate Igα-Igβ-bearing B cells in animals models, in which the animal is engrafted with B cells bearing the target Igα-Igβ BCR. For example, a mouse, rat, or other laboratory animal can be engrafted with human B cells. Such animals can be xenograft animals, or immunodeficient animals that tolerate engraftment. SCID mice and other immune deficient animals permit xenotransplantation of a human B cell-lineage tumor. Such mammals provide excellent models for screening or testing drug candidates for human therapeutics.

Preferably, the animal is transplanted with a cell line corresponding to a CLL or other B cell lineage tumor (in which the B cells express BCR). The cell line can be a primary cell line, freshly derived from a patient. In an alternative embodiment, the animals are injected with CLL cells. The animals may be injected subcutaneously or intraperitonially.

### In Vivo Testing Using Transgenic Animals

Transgenic animals, and preferably mammals, can be prepared for measuring the efficacy of B cell elimination with anti-Igα-Igβ antibodies. Preferably, for evaluating compounds for use in human therapy, the animals are “humanized” with respect to Igα and Igβ. The term “transgenic” usually refers to animal whose germ line and somatic cells contain the transgene of interest, i.e., Igα and/or Igβ. However, transient transgenic animals can be created by the ex vivo or in vivo introduction of an expression vector of the invention. Both types of “transgenic” animals are contemplated for use in the present invention, e.g., to evaluate the effect of an antibody on B cell elimination.

Thus, human Igα and/or Igβ “knock-in” mammals can be prepared for evaluating the molecular biology of this system in greater detail than is possible with human subjects. Although rats and mice, as well as rabbits, are most frequently employed as transgenic animals, particularly for laboratory studies of protein function and gene regulation in vivo, any animal can be employed in the practice of the invention.

A “knock-in” mammal is a mammal in which an endogenous gene is substituted with a heterologous gene (Roemer et al., New Biol. 1991, 3:331). Preferably, the heterologous gene or regulation system is “knocked-in” to a locus of interest, either the subject of evaluation of expression (in which case the gene may be a reporter gene; see Elefanty et al., Proc Natl Acad Sci USA, 1998, 95:11897) or function of a homologous gene, thereby linking the heterologous gene expression to transcription from the appropriate promoter. This can be achieved by homologous recombination, transposon (Westphal and Leder, Curr Biol 1997, 7:530), using mutant recombination sites (Araki et al., Nucleic Acids Res 1997, 25:868) or PCR (Zhang and Henderson, Biotechniques 1998, 25:784; see also, Coffman, Semin. Nephrol. 1997, 17:404; Esther et al., Lab. Invest. 1996, 74:953; Murakami et al., Blood Press. Suppl. 1996 2:36).

Generally, for homologous recombination, the DNA will be at least about 1 kilobase (kb) in length and preferably 3-4 kb in length, thereby providing sufficient complementary sequence for recombination when the construct is introduced. Transgenic constructs can be introduced into the genomic DNA of the ES cells, into the male pronucleus of a fertilized oocyte by microinjeciton, or by any methods known in the art, e.g., as described in U.S. Pat. Nos. 4,736,866 and 4,870,009, and by Hogan et al., Transgenic Animals: A Laboratory Manual, 1986, Cold Spring Harbor. A transgenic founder animal can be used to breed other transgenic animals; alternatively, a transgenic founder may be cloned to produce other transgenic animals.

### Pharmaceutical Compositions

Antibodies of the present invention, or vectors encoding them, may be formulated into a pharmaceutical composition. The pharmaceutical composition comprises a pharmaceutically effective amount of the antibody of the present invention. The pharmaceutical composition also typically comprises a pharmaceutically acceptable carrier (or dosing vehicle), such as ethanol, glycerol, water, and the like. Examples of such carriers and methods of formulation are described in Remington's Pharmaceutical Sciences, 18th edition (1990), Mack Publishing Company.

The pharmaceutical composition may also include other additives, such as a flavorant, a sweetener, a preservative, a dye, a binder, a suspending agent, a colorant, a disintegrant, an excipient, a diluent, a lubricant, a plasticizer, or any combination of any of the foregoing. Suitable binders include, but are not limited to, starch; gelatin; natural sugars, such as glucose and beta-lactose; corn sweeteners; natural and synthetic gums, such as acacia, tragacanth, and sodium alginate; carboxymethylcellulose; polyethylene glycol; waxes; and the like. Suitable lubricants include, but are not limited to, sodium oleate, sodium stearate, magnesium stearate, sodium benzoate, sodium acetate, sodium chloride and the like. Suitable disintegrators include, but are not limited to, starch, methyl cellulose, agar, bentonite, xanthan gum and the like.

The pharmaceutical compositions may be formulated as unit dosage forms, such as tablets, pills, capsules, boluses, powders, granules, sterile parenteral solutions or suspensions, elixirs, tinctures, metered aerosol or liquid sprays, drops, ampoules, autoinjector devices or suppositories for oral, parenteral, intranasal, sublingual or rectal administration, or for administration by inhalation or insufflation. The unit dosage form may be in a form suitable for once-weekly or once-monthly administration, such as, an insoluble salt of the compound, e.g., a decanoate salt, adapted to provide a depot preparation for intramuscular injection.

Solid unit dosage forms may be prepared by mixing the compound of the present invention with a pharmaceutically acceptable carrier and any other desired additives to form a solid preformulation composition. Examples of suitable additives for solid unit dosage forms include, but are not limited to, starches, such as corn starch; lactose; sucrose; sorbitol; talc; stearic acid; magnesium stearate; dicalcium phosphate; gums, such as vegetable gums; and pharmaceutical diluents, such as water. The solid preformulation composition is typically mixed until a homogeneous mixture of the compound of the present invention and the additives is formed, i.e., until the compound is dispersed evenly throughout the composition, so that the composition may be readily subdivided into equally effective unit dosage forms. The solid preformulation composition is then subdivided into unit dosage forms of the type described above containing from about 0.01 to about 50 mg of the compound of the present invention.

Tablets or pills can be coated or otherwise compounded to form a unit dosage form which has prolonged action, such as time release and sustained release unit dosage forms. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release.

Liquid unit dosage forms include, but are not limited to, aqueous solutions, suitably flavoured syrups, aqueous or oil suspensions, and flavoured emulsions with edible oils, such as cottonseed oil, sesame oil, coconut oil or peanut oil, as well as elixirs and similar pharmaceutical vehicles. Suitable dispersing and suspending agents for aqueous suspensions include, but are not limited to, synthetic and natural gums, such as tragacanth, acacia, alginate, dextran, sodium carboxymethylcellulose, methylcellulose, polyvinyl-pyrrolidone and gelatin.

Suitable pharmaceutically acceptable carriers for topical preparations include, but are not limited to, alcohols, aloe vera gel, allantoin, glycerine, vitamin A and E oils, mineral oil, PPG2 myristyl propionate, and the like. Such topical preparations may be liquid drenches, alcoholic solutions, topical cleansers, cleansing creams, skin gels, skin lotions, and shampoos in cream or gel formulations (including, but not limited to aqueous solutions and suspensions). Typically, these topical preparations contain a suspending agent, such as bentonite, and optionally, an antifoaming agent. Generally, topical preparations contain from about 0.005 to about 10% by weight and preferably from about 0.01 to about 5% by weight of the compound, based upon 100% total weight of the topical preparation.

Pharmaceutical compositions of the present invention for administration parenterally, and in particular by injection, typically include an inert liquid carrier, such as water; vegetable oils, including, but not limited to, peanut oil, cotton seed oil, sesame oil, and the like; and organic solvents, such as solketal, glycerol formal and the like. A preferred liquid carrier is vegetable oil. These pharmaceutical compositions may be prepared by dissolving or suspending the compound of the present invention in the liquid carrier. Generally, the pharmaceutical composition for parenteral administration contains from about 0.005 to about 10% by weight of the compound of the present invention, based upon 100% weight of total pharmaceutical composition. Injection may be, for example, intramuscular, intraruminal, intratracheal, or subcutaneous.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The pharmaceutical composition of the present invention may be administered to an animal, preferably a human being, in need thereof to treat a condition of inappropriate B cell activation.

The antibodies of the present invention may be administered alone at appropriate dosages defined by routine testing in order to obtain optimal B cell elimination. In addition, co-administration or sequential administration of other active agents may be desirable.

The daily dosage of the compounds of the present invention may be varied over a wide range from about 0.01 to about 1,000 mg per patient, per day. For oral administration, the pharmaceutical compositions are preferably provided in the form of scored or unscored tablets containing 0.01, 0.05, 0.1, 0.5, 1.0, 2.5, 5.0, 10.0, 15.0, 25.0, or 50.0 milligrams of the compound of the present invention for the symptomatic adjustment of the dosage to the patient to be treated. An effective amount of the compound is ordinarily supplied at a dosage level of from about 0.0001 mg/kg to about 100 mg/kg of body weight per day. More preferably, the amount is from about 0.001 mg/kg to 10 mg/kg of body weight per day. The dosage amount maybe adjusted when combined with other active agents as described above to achieve desired effects. On the other hand, unit dosage forms of these various active agents may be independently optimized and combined to achieve a synergistic result wherein the pathology is reduced more than it would be if either active agent were used alone.

Advantageously, the pharmaceutical compositions may be administered in a single daily dose, or the total daily dosage may be administered in divided doses of two, three or four times daily. Furthermore, the pharmaceutical compositions can be administered in intranasal form via topical use of suitable intranasal vehicles, or via pulmonary routes, using aerosols.

For combination treatment with more than one active agent, where the active agents are in separate dosage formulations, the active agents can be administered concurrently, or they each can be administered at separately staggered times.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound thereof employed. A physician or veterinarian of ordinary skill can readily determine and prescribe the effective amount of the drug required to prevent, counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug.

The compound may also be administered as an additive to the feed by simply mixing the compound with the feedstuff or by applying the compound to the surface of animal feed. Alternatively, the compound may be mixed with an inert carrier and the resulting composition may then either be mixed with the feed or fed directly to the animal. Suitable inert carriers include, but are not limited to, corn meal, citrus meal, fermentation residues, soya grits, dried grains, and the like. The compound may be intimately mixed with the inert carrier by grinding, stirring, milling, or tumbling such that the final composition contains from 0.001 to 5% by weight of the compound, based upon 100% total weight of composition.

### Treatment of B Cell Lymphomas

The present invention provides strategies for eliminating B cells that exhibit inappropriate activity from a system. B cells that exhibit inappropriate activity include B cell malignancies and neoplasms such as, but not limited to, CLL, Burkitt's lymphoma, plasmacytomas, B cell leukemias, and B cell malignancies that express BCRs on the cell surface. The present invention is directed the treatment of non-solid tumors, particularly leukemia. In a preferred embodiment of the present invention, the non-solid tumor is chronic lymphocytic leukemia (CLL).

The phrase “therapeutically effective” or “therapeutic” is used herein to mean to reduce by at least about 15 percent, preferably by at least 50 percent, more preferably by at least 90 percent, and most preferably eliminate, a clinically significant deficit in the activity, function and response of the subject. Alternatively, a therapeutically effective amount is sufficient to cause an improvement in a clinically significant condition in the subject.

For example, a therapeutic effect is achieved by inhibiting B cell proliferation when cellular apoptisis is greater with an anti-Igα-Igβ antibody than in the absence of the antibody. Such effects include reducing tumor size, eliminating metastasises, increasing time to recurrence, or increasing survival. Antibodies can be provided to subjects in pharmaceutically acceptable formulations and via routes of administration, as described above. Patients may be monitored at weekly, biweekly, or monthly intervals. Blood samples may be obtained to determine the levels of CLL tumor cells that are present.

### Gene Therapy

In a specific embodiment, vectors comprising a sequence encoding a protein, including, but not limited to, Igα, Igβ, or Igα-Igβ heterodimer, are administered to treat or prevent a disease or disorder associated with inappropriate B cell activity. In this embodiment of the invention, the therapeutic vector encodes a sequence that produces the antibody of the invention.

Any of the methods for gene therapy available in the art can be used according to the present invention. Exemplary methods are described below.

For general reviews of the methods of gene therapy, see, Goldspiel et al., Clinical Pharmacy, 1993, 12:488-505; Wu and Wu, Biotherapy, 1991, 3:87-95; Tolstoshev, Ann. Rev. Pharmacol. Toxicol., 1993, 32:573-596; Mulligan, Science, 1993, 260:926-932; and Morgan and Anderson, Ann. Rev. Biochem., 1993, 62:191-217; May, TIBTECH, 1993, 11:155-215. Methods commonly known in the art of recombinant DNA technology that can be used are described in Ausubel et al, (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al., (eds.), 1994, Current Protocols in Human Genetics, John Wiley & Sons, NY. Vectors suitable for gene therapy are described above.

In one aspect, the therapeutic vector comprises a nucleic acid that expresses an antibody of the invention in a suitable host. In particular, such a vector has a promoter operationally linked to the coding sequence for the antibody. The promoter can be inducible or constitutive and, optionally, tissue-specific. In another embodiment, a nucleic acid molecule is used in which the protein coding sequences and any other desired sequences are flanked by regions that promote homologous recombination at a desired site in the genome, thus providing for intrachromosomal expression of the antibody (Koller and Smithies, Proc. Natl. Acad. Sci. U.S.A, 1989, 86:8932-8935; Zijlstra et al., Nature, 1989, 342:435-438).

Delivery of the vector into a patient may be either direct, in which case the patient is directly exposed to the vector or a delivery complex, or indirect, in which case, cells are first transformed with the vector in vitro then transplanted into the patient. These two approaches are known, respectively, as in vivo and ex vivo gene therapy.

In a specific embodiment, the vector is directly administered in vivo, where it enters the cells of the organism and mediates expression of the protein. This can be accomplished by any of numerous methods known in the art, including, by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont); or coating with lipids or cell-surface receptors or transfecting agents, encapsulation in biopolymers (e.g., poly-S-1-64-N-acetylglucosamine polysaccharide; see, U.S. Pat. No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (see, e.g., Wu and Wu, J. Biol. Chem., 1987, 62:4429-4432), etc. In another embodiment, a nucleic acid ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188). Alternatively, the nucleic acid can be introduced intracellularly and incorporated within host cell DNA for expression by homologous recombination (Koller and Smithies, Proc. Natl. Acad. Sci. USA, 1989, 86:8932-8935; Zijlstra, et al., Nature, 1989, 342:435-438). These methods are in addition to those discussed above in conjunction with “Viral and Non-viral Vectors”.

Alternatively, antibody molecules also can be administered, for example, by expressing nucleotide sequences encoding single-chain antibodies within the target cell population by utilizing, for example, techniques such as those described in Marasco et al. (Proc. Natl. Acad Sci. USA, 1993, 90:7889-7893).

The form and amount of therapeutic nucleic acid envisioned for use depends on the type of disease and the severity of the desired effect, patient state, etc., and can be determined by one skilled in the art.

### EXAMPLES

The present invention will be better understood by reference to the following Examples, which are provided as exemplary of the invention, and not by way of limitation.

### Example 1

### Igα-Igβ Antigen Production

The membrane external domains of human Igα (encoding amino acid residues 1-112) and Igβ (encoding amino acid residues 1-129) are amplified by PCR (Stratagene). Fc region of human IgG1 is PCR amplified from a human cDNA plasmid. Each Igα and Igβ PCR product and the IgG1 Fc product, are digested and ligated into the pcDNA1 expression vector. CHO cells are transformed with these fusion constructs (pcDNA-Igα Fc and pcDNA IgβFc) and reduced to express the fusion protein. The fusion proteins are purified by protein A affinity chromatography. The Fc region is removed by proteolytic cleavage, Igα and Igβ polypeptides purified from the Fc cleavage product by gel permeation chromatography or protein A affinity chromatography, or both. Igα and Igβ are mixed under oxidizing conditions to permit desulfide crosslinking. The heterogenous (α-α, β-β, and α-β) mixture is used for immunization.

### Example 2

### Anti-Igα-Igβ Antibody Production

The purified Igα-Igβ heterodimer/hemodimer mixture is prepared in complete Freund's Adjuvant at a concentration of 10 μg protein per mL and injected into XenoMice™. Mice are boosted three times at one week intervals with the same concentration of antigen in Incomplete Freund's Adjuvant. Spleen cells from XenoMice™ with a robust antiserum response are fused with myeloma cells using standard fusion protocols. Hybridomas are selected and screened from finding to Igα and Igβ to produce monoclonal antibodies using protocols that are known in the art.

Positive hybridomas are cloned by limiting dilution. Monoclonal antibodies from hybridoma clones are further tested for reactivity with human B cell lines expressing human Ig Anti-Igβ and anti-Igα antibodies are screened for specificity by staining available human cell lines. These cell lines include, but are not limited to, fibroblasts, epithelial cells, red blood cells, platelets, macrophages, mast cells, polymorphonuclear leukocytes, and neuronal cell lines. Specificity also is tested by tissue staining with human tissue samples. B cells serve as positive controls in these studies.

Those antibodies that stain human B cells are further tested for the ability to induce B cell apoptosis in vitro and for selectivity. Antibodies are selected for further testing based on their biological activity and lack of crossreactivity. Active antibodies are defined as those antibodies that induce B cell apoptosis, as measured by standard apoptosis assays, or induce B cell proliferation, as measured by standard in vitro proliferation assays.

Antibodies are further tested for efficacy on CLL cell lines and then on primary CLL tumor cells from patients in vitro. CLL cells are assayed for induction of apoptosis.

### Example 3

### In Vivo Animal Model of CLL

Groups of SCID mice are used in an in vivo model by transplanting high-stage peripheral blood mononuclear cells into the peritoneal cavity of the mice. A second group of SCID mice are treated by injecting CLL cells subcutaneously. Tumor cells are then permitted to double in size at least once. Animals then are either injected subcutaneously or intraperitoneally with anti-Igα-Igβ antibodies in saline. One control group of mice are injected with saline only. At weekly intervals, the mice receive a boost injection. Mice are bled prior to each injection and tested to determine the level of CLL cells present. Additionally, the size of the lymph nodes are monitored assess proliferation of B cells in vitro. Groups of mice are sacrificed following the final boost and spleen and lymph node lymphocytes tested for level of CLL cells present.

### Example 4

### Human Clinical Trial Protocol

Patients with CLL who have not responded to standard therapies or have relapsed following standard therapies are randomized into double-blinded treatment and control groups. Patients in the treatment groups receive human anti-Igα-Igβ antibodies at 1, 50, and 500 mg doses, intravenously at weekly intervals. Control (three groups) patients receive vehicle containing an irrelevant antibody with the same framework and constant regions and at the same dosage as the treatment groups. Blood samples are obtained one week after each dose to determine levels of CLL tumor cells present. Additionally, the size of the lymph nodes are monitored assess proliferation of B cells. Patients are monitored for response and for time of progression of the disease. Time to death also is recorded. Comparison of each test and control groups outcomes, and across the dosage groups, with statistical analysis of the outcomes establish that the anti-Igα-Igβ antibody is therapeutically effective.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that values are approximate, and are provided for description.

Patents, patent applications, publications, procedures, and the like are cited throughout this application, the disclosures of which are incorporated herein by reference in their entireties.

What is claimed is:

1. An anti-Igα-Igβ antibody which binds an external membrane domain binding region of a mammalian Igα-Igβ complex, wherein binding of the antibody to a B cell induces B cell elimination.

2. The anti-Igα-Igβ antibody of claim 1, which is human.

3. The anti-Igα-Igβ antibody of claim 1, which is humanized.

4. The anti-Igα-Igβ antibody of claim 1, wherein an epitope recognized by the antibody is an external membrane domain binding region having a sequence corresponding to about amino acid residue 1 to about amino acid residue 112 of Igα with SEQ ID NO:1.

5. The anti-Igα-Igβ antibody of claim 1, wherein an epitope recognized by the antibody is an external membrane domain binding region having a sequence corresponding to about amino acid residue 1 to about amino acid residue 129 of Igβ with SEQ ID NO:3.

6. A nucleic acid encoding the anti-Igα-Igβ antibody of claim 1.

7. A nucleic acid encoding the anti-Igα-Igβ antibody of claim 3.

8. An expression vector comprising the nucleic acid of claim 6 operably associated with an expression control sequence.

9. An expression vector comprising the nucleic acid of claim 7 operably associated with an expression control sequence.

10. A host cell transfected with the expression vector of claim 8.

11. A host cell transfected with the expression vector of claim 9.

12. A method for producing an anti-Igα-Igβ antibody, which method comprises isolating the antibody from the host cell of claim 10 cultured under conditions that permit antibody expression.

13. A method for producing an anti-Igα-Igβ antibody, which method comprises isolating the antibody from the host cell of claim 11 cultured under conditions that permit antibody expression.

14. A pharmaceutical composition comprising a pharmaceutically effective amount of the antibody of claim 1, wherein the pharmaceutically effective amount is sufficient to induce B cell elimination, and a pharmaceutically acceptable carrier or excipient.

15. A method of eliminating a B cell, which method comprises contacting the B cell with the pharmaceutical composition of claim 14 effective to induce elimination of B cells.

16. A method for treating a condition of inappropriate B cell activity by inducing elimination of B cells of a subject in need of such treatment, which method comprises administering an amount of the pharmaceutical composition of claim 14 effective to induce elimination of B cells of the subject.

17. The method of claim 16, wherein the subject is suffering from a B cell lymphoma.

18. The method of claim 17, wherein the B cell lymphoma is chronic lymphocytic leukemia (CLL).

19. A method of eliminating tumor cells of a B cell lymphoma, which method comprises contacting the tumor cells with an amount of the antibody of claim 1 effective to eliminate the tumor cells.

20. The method of claim 19, wherein the B cell lymphoma is CLL.

21. A pharmaceutical composition comprising the expression vector of claim 10 in an amount effective to express a therapeutically effective amount of the antibody effective to eliminate B cells in vivo.

22. A pharmaceutical composition comprising the expression vector of claim 11 in an amount effective to express a therapeutically effective amount of the antibody effective amount of the antibody effective to eliminate B cells in vivo.

23. A method for treating a condition of inappropriate B cell activity by eliminating B cells of a subject in need of such treatment, which method comprises administering an amount of the pharmaceutical composition of claim 21 effective to eliminate B cells of the subject.

24. A method for treating a condition of inappropriate B cell activity by eliminating B cells of a human subject in need of such treatment, which method comprises administering an amount of the pharmaceutical composition of claim 22 effective to eliminate B cells of the subject.

25. A host cell transfected with an expression vector comprising a nucleic acid encoding Igα operably associated with an expression control sequence and transfected with an expression vector comprising a nucleic acid encoding Igβ operably associated with an expression control sequence.

26. A method for producing a Igα-Igβ heterodimer protein, which method comprises culturing the host cell of claim 25 under conditions that permit expression of a Igα-Igβ heterodimer protein.

27. The method of claim 26, wherein Igα and Igβ are each expressed as a fusion protein.

28. The method of claim 27, wherein each fusion protein independently is fused to Ig, a FLAG tag, or a HIS tag.

29. A method for producing anti-Igα-Igβ antibody, which method comprises immunizing an animal with an amount of Igα, Igβ, Igα and Igβ, or an Igα-Igβ heterodimer with an adjuvant to produce an anti-Igα-Igβ antibody, wherein the animal is a different species than the Igα-Igβ species.

30. The method of claim 29, wherein Igα and Igβ are expressed as a fusion protein.

31. The method of claim 30, wherein each fusion protein independently is fused to Ig, a FLAG tag, or a HIS tag.

32. The method of claim 29, wherein the animal is a mouse and the Igα-Igβ is human.

33. The method of claim 32, wherein the mouse is a xenograft mouse that has a human immune system.

34. The method of claim 32, which further comprises humanizing the antibody by inserting CDRs from the antibody generated in the mouse into a human antibody framework.

35. The method of claim 29, which further comprises screening the antibody for the ability to eliminate B cells.
